(19) United States
(12) Patent Application Publication
(10) Pub. No.: US 2004/0080418 A1
(43) Pub. Date: Apr. 29, 2004

(54) Method and device to prevent a person from using a drug

(76) Inventors: Carl Dahlborn, Lidingo (SE); Bertil G Guve, Stockholm (SE); Patrik Wahren, Djursholm (SE)

Correspondence Address: BIRCH STEWART KOLASCH & BIRCH, PO BOX 747, FALLS CHURCH, VA, 22040-0747, US

(21) Appl. No.: 10/332,851

(22) PCT Filed: Jul. 13, 2001
(86) PCT No.: PCT/SE01/01621

(30) Foreign Application Priority Data
Jul. 14, 2000 (SE) 0002672-4

(51) Int. Cl. G08B 23/00; A61B 5/08

(52) U.S. Cl. 340/573.1; 600/532

(57) ABSTRACT

The invention refers to a solution of the problem that during a longer time prevail upon alcoholics or other drug abusers to refrain from alcohol or other drug. Methods and devices to prevent that a person utilises a drug comprises that a punishing unit is fixed to the person in such a way that the punishing unit not without difficulty can be removed, that a sensing or test unit which according to predetermined criteria detects the existence of drug use by the person is provided outside the body of a person, and that the sensing or test unit is arranged to communicate with the punishing unit and control it. The punishing unit can comprise means for brining out negative reactions in the person, for example in the form of an electric stimulator.

The present invention refers to a method and a device to prevent a person from using alcohol or other drug. The invention in particular refers to a method that involves detection of the presence of a drug in the body of the person and that a punishing device is started in the presence of the drug. The detection of the drug either may be carried out by means of a device for sensing the presence of the drug or any substance or condition in the body associated with the drug. The detection can also be carried out by means of a test that reveals that the person is influenced by the drug. The punishing device may be constituted by a device that effects that negative reactions are obtained in the person or by a device that records or relays information about the condition of the person.

In a preferred embodiment a device according to the invention is divided into two units, whereby a first unit, further called sensing or test unit, includes sensing means or testing means and it is intended to be placed outside the body and may constitute an independent unit, while a second unit that preferably includes means to evoke negative reactions in the person, the punishing unit, preferably is intended to be permanently connected to the person so that it cannot easily be removed, for example that it is attached to an extremity or implanted. A communication link between the sensing or test unit and the punishing unit ensures that the information from the test unit can influence the function of the punishing unit. The negative reactions for instance can be accomplished by electric stimulation or dispensing a preparation in the body. Moreover, some of the units may contain means for recording and/or relaying information about the condition of the person.

Methods and devices according to the present invention are particularly adapted for use during a longer period to get alcoholics out of habit in order to completely prevent the use of alcohol, whereby the means for evoking the negative reactions normally never are activated since the knowledge of the effect of the device makes the person to completely relinquish alcohol.

Through WO 95/33445 is previously known an implantable device containing an alcohol sensor and electrical stimulator or preparation pump for bringing about negative reactions in a person. It is further known through the U.S. Pat. No. 4,003,379 (see in particular column 4, the second paragraph) to detect drug abuse in a person by sensing a combination of secondary conditions such as breathing and certain muscle movements in order to guide a treatment.

According to the present invention by separating the equipment according to the first-mentioned publication so that at least the drug sensing part is placed outside the body a simpler, cost efficient and more reliable equipment may be provided that is easier accessible for calibration and service and where the external part normally does not need to be miniaturised. The test unit according to the present invention may also be provided as an alternative to a plain sensing of the presence of the drug in the body to call for tests that the person has to perform and the results of which may give an indication of influence of a drug.

Thus, an object of the present invention is to provide a method and a device that constitutes an efficient hindrance for a drug abuser to exert his abuse. An object is also to provide an equipment that can be operative during long time and contain vital parts for the function which cannot be manipulated or removed by the addict. A further object is that the device shall not emit any preparation when a drug or the like is not sensed, whereby side effects of long term use of a chemical preparation is eliminated. Still an object is to utilise the knowledge of the addict of the function and the effect so that he(she) does not exert the abuse and the device is not activated. These and other objects are accomplished by providing a method and a device according to the invention the features stated in the subsequent claims.

### The invention will now be described in closer detail with reference to the drawing.

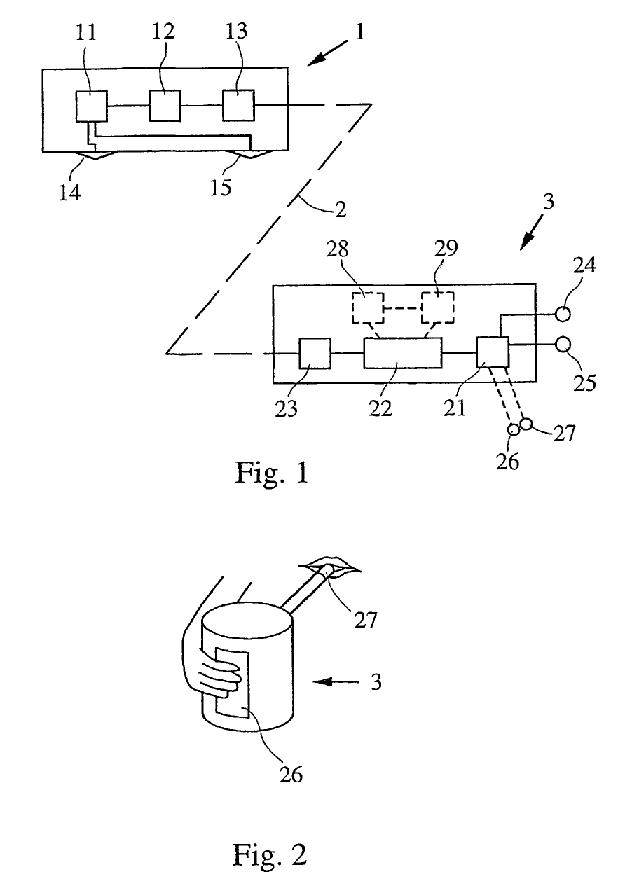

FIG. 1 shows diagrammatically an embodiment of a device according to the invention.

FIG. 2 shows how a closed circuit between the hand and mouth can be used in an expiration test.

The embodiment according to FIG. 1 includes a punishing unit 1, a sensing or test unit 2 and a communication link 3 between the units 1 and 2. The punishing unit is intended to be fixed to or implanted in the body of the person and can be any of the types more exactly described in the above mentioned patent publication WO 95/33445 and thus consists of an electric stimulator or a preparation pump which when they are activated are intended to evoke discomfort for the person who carries the punishing unit.

The punishing unit 1 according to FIG. 1 contains an electric stimulator 11 which is connected to at least two electrodes 14 and 15 which are placed on the top of the housing of the unit or at the ends of connection lines extending outside the housing so that they permanently can bear on the skin or in another way permanently may be connected to suitable portions of the body so that electric pulses or other signals generated and emitted by the stimulator 11 cause unpleasant sensations in the person carrying the unit. When the punishing unit is implanted one or all electrodes may be placed on the housing of the unit but one or several electrodes may also be connected through wires in or under the skin.

Preferably the punishing unit is intended to be placed on the skin in such a way that it not without difficulty can be removed by the user. This can technically be solved by a technique similar to that being used in so called electronic foot shackles. For example the unit may be fixed to an arm or a leg by means of a lockable strip around the extremity. The electrodes may be placed such that they bear on the skin under the punishing unit or by connection wires fixed to the skin at a distance from the punishing unit. The electrodes in both cases may be fixed by holes in the skin which holes can be made in a similar manner as that by perforation for jewellry, so called piercing.

The punishing unit 11 also comprises a first control unit 12 which controls the stimulator 11 so that it is started only when certain predetermined conditions are met. The control unit 12 preferably consists of electronic circuits and may comprise a microprocessor. In its simplest embodiment the control unit 12 only comprises a timer circuit or counter that emits a start signal to the stimulator after countdown by a definite time if it not before this time receives a signal for restart. The control unit 12 normally receives input signals from the second communication unit 23 in the sensing or test unit 2 via the communication link 3 to the first communication unit 13 in the punishing unit 1. If the sensing or test unit has sensed that no drug is present or performed tests that indicate this, then a signal is generated for restart to the timer circuit or the counter in the control unit 12.

Preferably, the communication link 3 and the first and second communication units 13 and 23 may operate with any type of wireless transmission that is suitable for short distance communication, for example radio, IR- or ultrasonic transmission of a previously known type.

In addition to the second communication unit 23 the sensing and test unit 2 comprises a second control unit 22, a detector unit 21 that is preferably linked to a first input/output means 24 and a second input/output means 25, additional input/output means or only one- may also be provided. The detector unit can operate according to two separate principles, either it can be intended to directly detect a drug or a substance related to this or a state in the body, whereby input/output means 24, 25 are constituted by one or more sensors, or else the detector unit can be provided to perform tests on which the person should react and perform predetermined actions.

A simple test that is initiated by the detector unit 21 is constituted by a reaction test, by which the person in response to lighting of a luminous point shall press a button. The input/output means 24 and 25 thus are constituted by a LED/lamp and a push-button respectively. Even sonic signals are applicable and the luminous point can flash with a certain frequency, whereby the flashing normally is easier to be aware of for an unaffected person. When the sensing or test unit 2 is of this type it is preferably intended to be carried as a wrist watch so that luminous points and push-buttons are readily visible and accessible. The unit can be programmed or controlled such that tests shall be carried out at randomly chosen occasions or with predetermined time intervals. When simpler tests are performed the person may perform a test series, whereby some fails are accepted without the punishing unit being activated.

In a preferred embodiment according to the invention a detector unit 21 is utilised where the alcohol concentration or any substance related to alcohol or other drug is detected in the expiration air, which can be carried out by means of a known apparatus. In this case co-operation of the person is required to perform the test, while the test result is an objective measuring value, why this test can be regarded to constitute a combination of both principles stated above. The sensing or test unit 2 can be implemented as a completely independent unit which however must be available for the person when a test is required. The first input/output means 24 is thereby constituted by the sensor to detect the substance and the second input/output means 25 is constituted by a sensor for sensing that a complete exhalation has been carried out.

In order to ensure that it is the person himself who performs the exhalation when detecting in the expiration air, there may be provided special means to monitor this. An example of this appears from FIG. 2. The detector unit 21 can further be provided with further input/output means 26, 27 in the form of electrodes at the end of the blowing mouthpiece which will be held in mouth and also on another part of the equipment which will be held in hand. Through sensing that a closed circuit is formed between the electrodes 26 and 27 and possibly also measuring of the impedance a relatively secure elimination of attempts to trick the apparatus can be accomplished. Even more advanced safety tests can be performed in a similar manner, for example comparison of EKG- or pulse curves or other physiological measurements. Similar control measurements can also be made in connection with other tests, push-buttons which are going to be used can be provided with electrodes.

Irrespective type of sensing or test unit 2 the second control unit 22 is arranged in response to an approved test from the detector unit to send a reset or inhibition signal to the punishing unit 1, alternatively in response to a rejected test to send an activating signal to the punishing unit 1. The first case corresponds to the one where the punishing unit comprises a timer or counter and activation of the punishing occurs automatically unless reset happens within a certain time, which automatic gives punishing if the sensor or test unit 2 is removed or manipulated. The second case demands that the punishing unit is arranged to be activated in response to a signal from the sensing or test unit 2, which gives protection against malfunctions but also suitably have to be combined with a “hand shaking procedure” between the units 1 and 2 with suitable intervals for function monitoring. All functions mentioned herein may simply be implemented with electronics which may be program controlled.

In order to monitor that the equipment works or the state of the person, either of the punishing unit 1 or the sensing or test unit 2 can also be provided with a telephone equipment 29 for communication with an alarm centre or other remotely located equipment or person. Via mobile telephony thereby actual status or data recorded in a recording unit 28 in the equipment may be accessed. The stimulator of the punishing unit can also be completed by or in certain cases substituted with recording of test results which indicate damage and automatic calling from the equipment when this occurs.

In certain cases it may be advantageous to combine the punishing unit and the sensing or test unit to a single unit that will be carried on the body and not without difficulty or damage can be removed by the bearer. Thereby no communication link is required with separate communication units and both control units can be combined. Even other embodiments of the invention are possible within the scope of subsequent claims.

1. A method for preventing that a person utilises a drug, characterised in
that a punishing unit comprising means for bringing about negative reactions in the person or other punishing action is fixed to the person in such a way that the punishing unit not without difficulty can be removed, and
that a sensing or test unit which after predetermined criteria detects existence of drug use of the person is arranged outside the body of the person, and
that the sensing or test unit is arranged to communicate with the punishing unit and control this so that a punishing action is initiated by a detected drug use.

2. A method according to claim 1, characterised in that means are provided to ensure that actions or measurements that are necessary for detection are carried out by or on the person him(her)self.

3. A method according to claim 1, characterised in that the punishing unit is locked on to the body of the person.

4. A device for preventing that a person uses a drug, characterised in
a punishing unit intended to be permanently connected to the person, whereby the punishing unit includes means to emit a substance stored in the device or electric pulses from a stimulator in the device with such effect and dosage that negative reactions are brought about in the person, alternatively means to perform other punishing action,
a sensing or test unit that includes means to detect if the person has used the drug in question through direct sensing or through tests that the person will react on,
that the sensing or test unit is formed as an independent unit or arranged such that it can be fastened at the outside the body,
that means are provided for communication between the punishing unit and the sensing or test unit whereby the punishing unit is activated by detection of presence of drugs by the sensing or test unit.

5. A device according to claim 4, characterised in that the punishing unit is designed such that it can be implanted in the person.

6. A device according to claim 4, characterised in that the punishing unit is arranged such that it can be fixed to the body of the person in such a way that it can not be removed by the person himself without substantial damage.

7. A device according to claim 4, characterised in that means are provided in order to attract attention of the person when detection with the sensing or test unit has to proceed, whereby the person has to perform actions which are necessary for the detection to be carried out, and that the punishing unit is activated if the person does not perform the actions.

8. A device according to claim 7, characterised in that the means to attract the attention of the person is constituted by a time controlled or randomly emitted signal and that the actions have to be carried out within a predetermined time after the signal has been emitted.

9. A device according to claim 7, characterised in that it in addition includes means to ensure that actions which are necessary for detection are carried out by the person him(her)self.

10. A device according to claim 9, characterised in that the means to ensure that actions which are necessary for detection are performed by the person him(her)self, when the action consists in that the person shall blow in a mouthpiece, consists of sensor means at the mouthpiece and on another part which by detection must be in contact with the body of the person, as well as measuring means in order to measure that both sensing means are in contact with the body of the same person, whereby the measuring means sense an electric quantity in a circuit between the sensing means.
